(19) United States
(12) Patent Application Publication
(10) Pub. No.: US 2006/0210961 A1
(43) Pub. Date: Sep. 21, 2006

(54) Method and system for analysing a liquid sample

(76) Inventors: Alastair Magnaldo, Connaux (FR); Thierry Davin, Lapalud (FR)

Correspondence Address: Thelen Reid & Priest, P O Box 640640, San Jose, CA, 95161-6400, US

(21) Appl. No.: 10/553,564

(22) PCT Filed: Dec. 15, 2004
(86) PCT No.: PCT/FR04/50693

§ 371 Date: Oct. 18, 2005

(30) Foreign Application Priority Data
Dec. 17, 2003 (FR) 0351095

(51) Int. Cl. C12Q 1/00; C12M 1/34

(52) U.S. Cl. 435/4; 435/287.1

(57) ABSTRACT

The present invention relates to a method for analyzing a liquid sample by injection of the latter into a reaction loop coupled with illumination means and detection means, which comprises the following steps:
filling a reaction loop (42) with a minimum volume of the sample to be analyzed, this reaction loop forming a transparent pipe with which detection means are coupled,
injecting a fixed volume of at least one reagent into the reaction loop (42),
detecting levels of filtered light by these detection means (41),
discharging reagents located in the reaction loop (42). The present invention also relates to a system for analyzing a liquid sample.

### TECHNICAL FIELD

The invention relates to a method and a system for analyzing a liquid sample.

The field of application of this invention is that of methods for analyzing liquids. More specifically, the invention is applied to automated analysis of flowing liquids or static liquids (taken samples).

### STATE OF THE PRIOR ART

Flux Injection Analysis (FIA), or analysis by injecting an analyte in the liquid flux of a carrier (or vector fluid in the following), concerns a family of analytical techniques, one of which is described in the referenced document [1] at the end of the description. A first common principle to all the analytical methods applied in FIA analysis is controlled dispersion of a liquid in a vector liquid flux. The dispersion combines diffusion effects and dilution effects during flow in a pipe of small diameter.

This dispersion notably occurs when a restricted area of a liquid present in a pipe at a given concentration is introduced into a vector liquid flux, by the difference in the flow velocities between the edges and the centre of the pipe. At the same time, the diffusion dilutes the extreme portions of the area thereby creating a concentration gradient, especially at the ends.

Understanding the dispersion phenomena and chemical reactions which are added to them, is still incomplete. However, most of the time, extensive understanding of the phenomena is not required by virtue of a second principle common to all the applied analytical methods in FIA analysis: the very good reproducibility. Indeed, before applying FIA analysis, it was often necessary to obtain, in the analytical method, complete chemical reactions in order to attain comparable reproducibilities. FIA analysis often does not leave sufficient time for complete reactions, but ensures an identical and reproducible reaction time for each analysis. Thus, each portion of the sample undergoes different treatments, but in a reproducible way among the samples. This is significant progress in automated analysis, notably because of the reduction of analysis times and reduction in the user's interventions.

The first applications of FIA analysis use a continuous flux in a single direction: an area of the sample is injected into the continuous flux of a vector fluid. Over time, the continuous flux creates the mixture allowing the reaction of the analytical method to occur in order to generate detectable species. As illustrated in FIGS. 1a and 1b, this technique requires a pump 10, a two-way injection valve 11, an on-line detector 12 and a reaction loop 13. This loop 13 consists of a pipe separating the injection valve 11 from the detector 12. Introduction of the sample to be analyzed is performed through inlet E. S represents the outlet for the effluents. Selection of the characteristics of the loop (dimensions and shape) depends on the relevant analytical method. The dead volume of the detector is sufficiently small for the requested resolution. The characteristics of the flow should be constant and reproducible. This often imposes a constant pipe diameter. The analytical frequency is imposed by the characteristics of the dispersion: it is thereby limited in order to avoid any contamination between successive samples.

FIGS. 1a and 1b represent a typical analytical sequence. In a first phase illustrated in FIG. 1a, the sample is passed into an injection loop until the contents of the injection valve 11 are representative of the latter. As illustrated in FIG. 1b, the injection valve 11 is then switched so as to allow the contents of the valve to be injected into the flux of reagent. The sample is then dispersed into the pipe 13 by the continuous flux so as to be detected in the detector 12 upon its passage.

The advantages of the FIA analysis technique as compared with prior techniques are a higher analytical frequency, a lower sample consumption and very good reproducibility. Its drawbacks are a larger consumption of reagents, a larger consumption of vector fluids and a high complexity of the sequences for methods requiring several treatment steps. The additional reagents required for the chemical reaction of the method are introduced through junctions in the vector fluid flux. Each reagent must thus be introduced through a bypass, and it therefore has a pumping unit which is specific to it.

FIA analysis as described above is widely used in automated analysis and contributes to the large majority of publications in this field. One of advancements from the FIA analysis technique is the analysis by sequential injection.

Sequential injection analysis or SIA analysis and FIA analysis have in common the dispersion principle and the reproducible handling of the fluids. SIA analysis further provides use of a bidirectional flux and periods for stopping the fluid. In addition, the two-position valve of FIA analysis is replaced with a multidirectional valve. SIA analysis may thus analyze solutions by using more complicated chemical methods, while keeping relatively reliable technological components.

FIGS. 2a to 2c, which illustrate a conventional SIA analysis system, illustrate a multidirectional valve 20, a mixing loop 21 and a detector 22, a retaining loop 23 and a bidirectional pump 24.

In general, analysis is carried out in three sequences. The first sequence is the filling of the system with vector solution, for example de-ionized water. The purpose of this sequence is to provide the system with an inert vector capable of transporting, even during flux inversions, the areas of the sample to be analyzed. The second sequence, as illustrated in FIGS. 2a and 2b, is alternate suction of sample areas, and of the required reagent(s) R for the analytical method as a train of areas, the whole being positioned in the retaining loop 23. The third sequence, as illustrated in FIG. 2c, is the dispersion of this train of areas in the mixing loop 21 followed by its passing in front of the detector 22. Forming a train of sample areas and of reagents only requires the use of a single pump 24, unlike the general case of FIA analysis. However, it should integrate additional constraints related to the bidirectional flux.

The advantages of SIA analysis as compared with FIA analysis, are the following: a more restricted number of technological components allowing more complicated methods to be applied, larger flexibility brought about by the possibility of inverting the flux and a greater optimization facility without requiring rewiring. However, the required volumes, notably of vector fluid, are large: typically 10 to 100 times larger than the volumes of reagents.

More recently, a possibility of sequential analysis without any vector solution analysis by CSIA (carrier-less sequential injection analysis or analysis by sequential injection without any carrier) has been suggested. CSIA analysis, for example described in the referenced document [2], includes the advantages of SIA analysis, including the small number of technological components, and avoids the potential drawbacks of the use of a vector fluid which are i.a. a high volume of analytical effluents related to the multiplication factor between the volumes of reagents and of vector solution.

A conventional CSIA analysis system is analogous to the system illustrated in FIGS. 2a to 2c. However, the analytical sequence is different. It is generally carried out according to the following steps: the retained loop 23 is filled with analyte by suction with the pump 24. A portion of the analyte is pumped back towards the mixing loop 21 and the detector 22. Suction of the analyte is completed. Next, the reagents are sucked up by switching over the multiport valve 20. By again switching this valve 20, the pump 24 is able to pump back the reagent and the analyte successively into the mixing loop 21 and the detector 22.

As compared with SIA analysis, elimination of the vector fluid results in the use of a larger analyte volume and in retention in a sufficiently voluminous loop.

Application of titration analytical methods (or volumetric analyses) by these controlled dispersion techniques requires the use of additional technical components. As illustrated in FIG. 3a, a technical solution consists of using a mixing chamber 30, located between the injection area 31 and the detector 32, the pump being referenced as 33. When a constituent is added to another one already present in the mixing chamber 30, a concentration gradient is obtained, allowing titration, at the outlet of this chamber 30, before passing in front of the detector 32. Another solution consists of using two pumps with variable flow rates in a sufficiently accurate way: one pump delivering the analyte, the other one delivering the titrating agent. A reaction loop performs partial or complete mixing of the solutions before passing in front of an on-line measuring cell. Titration is then carried out by establishing a gradient: the flow rate (or the concentration) of the titrating agent (or of the analyte) continuously varies over time, other properties of the mixture being kept constant. Such a solution, requiring successive individual steps, is time-consuming. The large number of individual measurements does not actually make it a continuous method.

Certain solutions describe continuous titration techniques by injection of the titrating agent at geometrical locations defined along a capillary, in which the analyte flows permanently. As described in the referenced document [3] and as illustrated in FIG. 3b, the analyte flowing in a capillary entering at E, receives at each injection location a flow rate of titrating agent T. After each consecutive addition and after its complete chemical reaction, the state of the mixture is measured by a detector 35. The consecutive additions are continued until depletion of the analyte. Pre-dilution is required in order improve accuracy.

The volumes of the analyte and of the liquid effluents may be of great size, notably in the case of samplings which may pose risks for humans, like radioactive or biological solutions. More generally, this may be the case of liquid solutions to be analyzed issued from a pre-treatment, for example from a concentration, separation or any chemical operation, which cannot be performed at a larger scale. This may also be the case of solutions of any kinds issued from submillimetric devices including microfluidic chips.

To summarize, often the analytical systems by FIA analysis cannot be used because of their large consumption of vector liquid and reagent. The analytical systems by SIA and CSIA analysis cannot be used, not only because of their large consumption of fluids in general, but also because of the size of the volumes and lengths of the retaining and reaction loops, the difficulty in making them compatible with the constraints required by most methods for manufacturing miniaturized circuits.

Further, the analytical systems by FIA, SIA or CSIA analyses require the use of valves, which is a penalty when miniaturization of the analysis is sought. Indeed, the setting-up of valves in microfluidic circuits requires an additional non-trivial technological step. SIA and CSIA analyses require the use of a bidirectional pump, which may pose certain concerns, notably degassing causing the formation of gas bubbles, greatly perturbing the reproducibility of the analyses especially when achievement of miniaturization is sought. The FIA, SIA and CSIA analyses require the injection of a sample in a reproducible way, notably as regards its volume, which is a source of analytical drifts.

Finally, when the analytical method requires titrations, adjunction of a mixing chamber is generally incompatible with the set purpose of miniaturization.

The object of the invention is to provide a technical solution to the problems raised above, by proposing an improved automatable analytical method by using less volume of reagents and analyte, in the absence of a vector fluid, with which volumetric analyses may be carried out, notably on a continuous flux, all of this over lengths and in volumes compatible with the techniques for manufacturing miniaturized fluidic circuits.

### DISCUSSION OF THE INVENTION

The invention relates to a method for analyzing a liquid sample by injection of the latter in a reaction loop coupled with illumination means and detection means, characterized in that it comprises the following steps:

filling a reaction loop with a minimum volume of the sample to be analyzed, this reaction loop forming a transparent pipe with which detection means are coupled,

injecting a fixed volume of at least one reagent into the reaction loop,

for example detecting levels of filtered light by these detection means,

discharging reagents located in the reaction loop.

Advantageously, a concentration gradient is detected in the reaction loop. The reaction loop may be a transparent capillary or a microfluidic channel. Discharge of the reagents located in the reaction loop may be performed by means of the remaining sample. It may also be performed by means of the next sample.

Advantageously, the sample flux is not interrupted, which allows continuous analysis. Fixed volumes of reagents may be successively injected during predefined time intervals. A series of reagent pulses may thereby be achieved with flow rate of the order of 10 to 1,000 μL.min⁻¹ followed by a waiting time. Linear detection may be performed along the reaction loop so that it is possible to obtain a space and time plot of the reactions in the set: reaction loop+detection means. Point detection may also be performed in a location of the reaction loop so that it is possible to obtain a time plot of the reactions in a location of the set: reaction loop+detection means. In this case, a point sensor may be used, capable of moving along the reaction loop.

The invention also relates to a system for analyzing a liquid sample comprising a reaction loop between this sample introduced through an inlet E and at least one reagent, and detection means, characterized in that the reaction loop consists of a transparent pipe and in that said system comprises a push-syringe, the outlet of which is connected to the reaction loop allowing doses of said at least one reagent to be delivered into this loop, and illumination means with which this reaction loop may be illuminated so that the detection means record levels of light transmitted through said loop after filtering.

The transparent pipe may be a transparent capillary or a microfluidic channel. The detection means may comprise a diode array or two optical fibers positioned on either side of the reaction loop. Advantageously, the sample may be introduced therein with a peristaltic pump. Advantageously, a microvalve may be positioned upstream from the point of introduction of the sample into the reaction loop. A T-shaped branch is respectively connected to the sample inlet E, to the push-syringe, and to the reaction loop.

Advantageously, the invention describes an analysis technique which requires neither vector fluid, nor retaining loop, nor inevitably a valve, nor inevitably a bidirectional pump. The reaction loop is also reduced in volume. This method does not require that the volume of the sample be known or measured. Its flow rate is also without any consequence on the measurement: the sample may be introduced ad hoc randomly as a flow, continuously and/or by gravity or by capillarity. This method also allows continuous and on-line titration of the solution, but also batchwise, with injection of reagents at a single flow rate.

During an analysis, the sample may be directly introduced into the device by a pump or gravity flow. It is neither necessary to know the volume of the sample, nor to strictly control its flow rate. The reaction loop and the detection areas may thereby be areas of continuous flow of the analyte. When it is desired to proceed with an analysis, the analyte flux may possibly be stopped by a simple valve. The fixed volume of reagent, perfectly under control, may be introduced at a given rate so that a concentration gradient of the reagent is established in the analyte. A waiting time is often desirable so that diffusion homogenizes at least partially the solution along the section of the channel. In this way, it is possible to inject, according to well-established timings and volumes, one after the other, other reagents or again the initial reagent so that it is possible to have at the detector not only a reproducible mixture of different reagents, but also mixing gradients of the analyte in the reagents. It is also possible by accurately actuating the pump with low flow rates to cause the mixing gradient established in the reaction loop to pass in front of a point detector, continuously over time. The result may then be expressed by the time lapse required for the detector to provide a value set beforehand. The set of on-line detectors may for example be a set of conductivity meters, potentiometers, and a linear CCD sensor, which for example provide a position corresponding to the detection of a given level of a parameter, for example the one corresponding to neutralization of an acid by a base, with which, by prior calibration, the contents of an element to be analyzed may be obtained. The point sensor may also be mobile along the reaction loop, for example, a set of optical fibers mounted on a stepping motor.

### BRIEF DESCRIPTION OF THE DRAWINGS

FIGS. 1a and 1b illustrate a typical analytical technique by FIA analysis.

FIGS. 2a-2c illustrate a typical analytical technique by SIA analysis.

FIG. 3a illustrates an analytical technique allowing titration by obtaining a concentration gradient over time.

FIG. 3b illustrates an analytical technique allowing continuous titration.

FIGS. 4a and 4b illustrate a first exemplary embodiment of the method of the invention.

FIGS. 5a and 5b illustrate a second exemplary embodiment of the method of the invention.

FIG. 6a illustrates a typical response from a diode array during a dosage according to the technique described in the second example.

FIG. 6b illustrates a typical curve obtained by the technique described in the second example, illustrating the position of the change of color of the dye depending on the acidity without loading the analyte.

FIGS. 7a and 7b illustrate a third exemplary embodiment of the method of the invention.

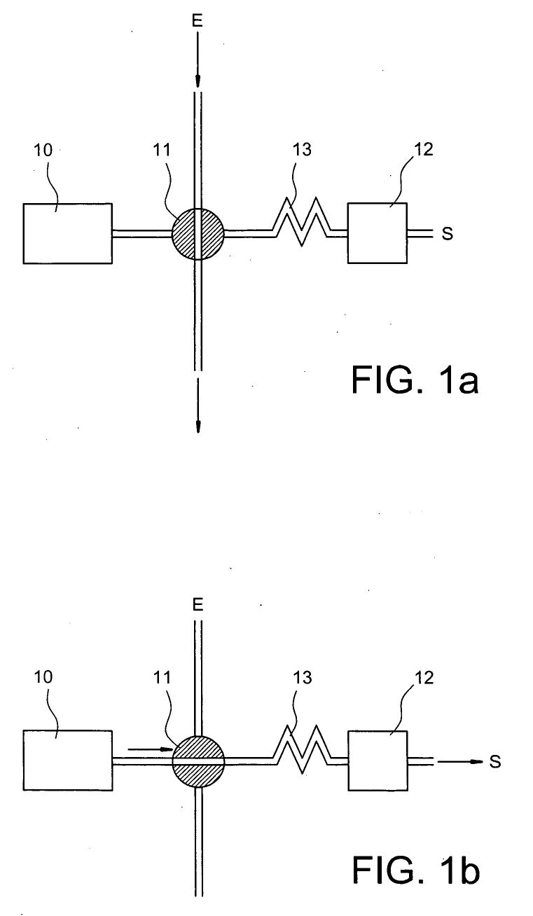

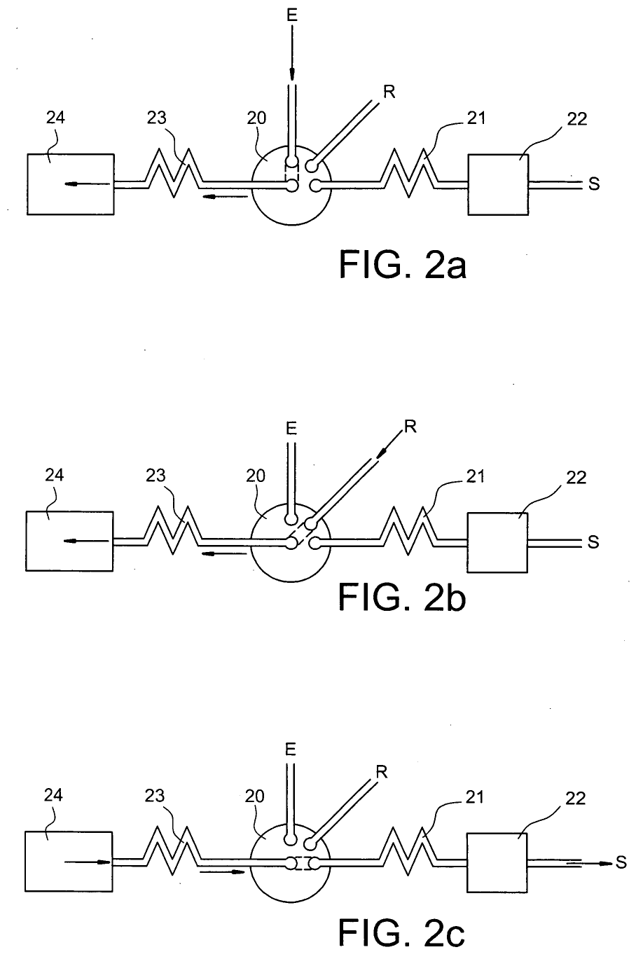

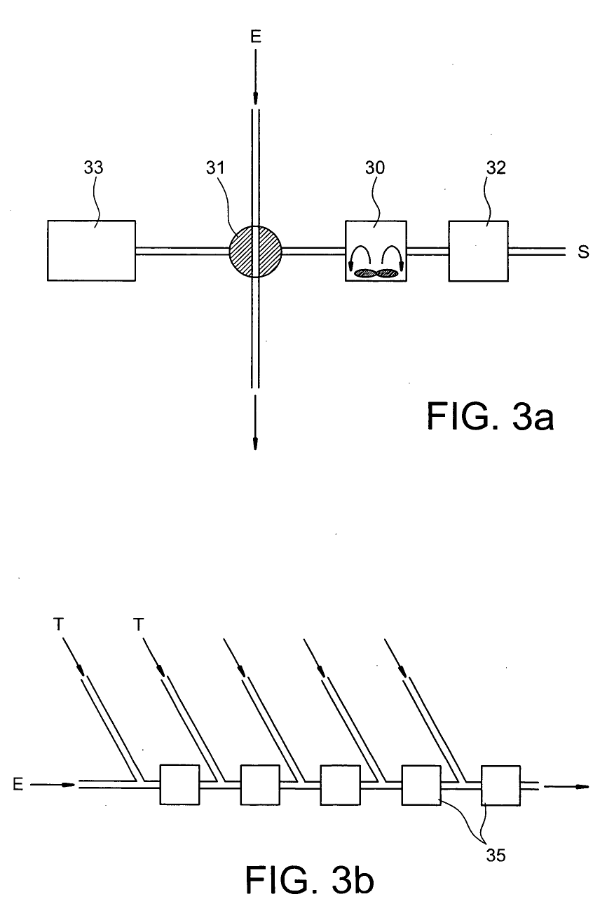

### DETAILED DISCUSSION OF PARTICULAR EMBODIMENTS

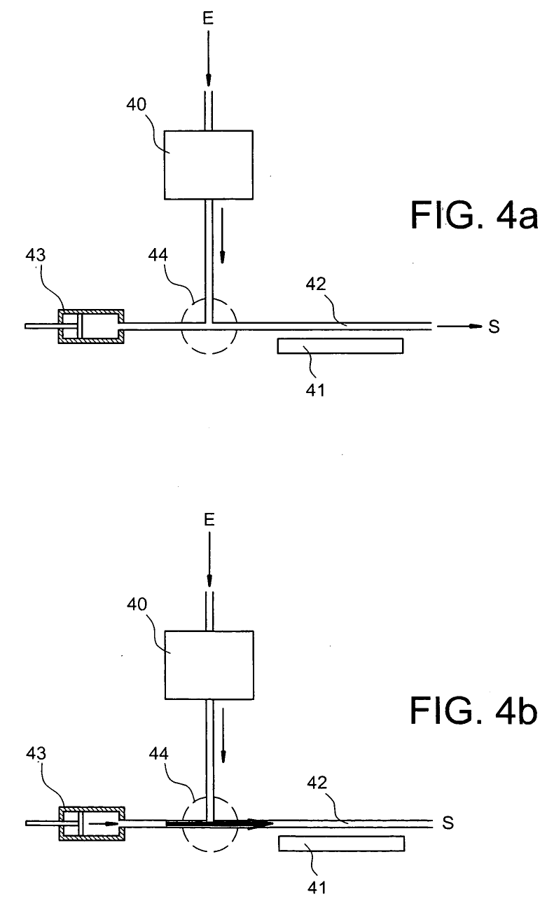

As illustrated in FIG. 4a for example, the present invention relates to a system for analyzing a liquid sample comprising a reaction loop 42, which consists of a transparent pipe, for example a transparent capillary or a microfluidic channel, between this sample entered at E and at least one reagent. With a push-syringe 43, the outlet of which is connected to the reaction loop 42, it is possible to deliver doses of said at least one reagent into the reaction loop. A T-shaped branch 44 allows the sample and the reagent(s) to be introduced into the reaction loop 42. Illumination means, for example a light-emitting diode, allow the reaction loop 42 to be illuminated, so that the detection means 41, for example a diode array, may record levels of light transmitted through said loop after filtering, these levels being representative of the characteristics of the sample, revealed by the mixture of the latter with the reagent(s).

The method of the invention comprises the following steps:

filling the reaction loop 42 with a minimum volume of the sample to be analyzed,

injecting a fixed volume of at least one reagent into the reaction loop 42,

detecting levels of filtered light by the detection means 41,

discharging the reagents located in the reaction loop 42.

Three exemplary embodiments according to the invention will now be examined.

### FIRST EXAMPLE

### Acid-Base Dosage

In this first example, as illustrated in FIGS. 4a and 4b, the sample is introduced through a peristaltic pump 40. The detector 41 is a diode array aligned on the reaction loop 42, which in this example is a transparent capillary, but it may also be a microfluidic channel. A dye, bromothymol blue (BBT), is diluted in a base (NaOH) contained in the syringe of a push-syringe 43.

The push-syringe 43 provided with a typically 100 to 500 μl syringe, is capable of delivering doses of the order of 1 μl in a sufficiently accurate way. The syringe at the output is coupled with the capillary 42. The sample issued from the peristaltic pump 40 encounters at a T-shaped branch 44, the capillary 42 fixed at the outlet of the push-syringe 43. This branch 44 is made by microfluidic manufacturing techniques. The capillary 42 at the outlet of the T branch 44, forming the reaction loop of the FIA and SIA analytical methods, has an inner diameter of 100 to 500 μm. Its length is from 0.5 to 10 cm.

The diode array 41 is illuminated by a light-emitting diode, not shown in FIGS. 4a and 4b. A filter, not shown, allows the blue shade of BBT in a more basic medium to be clearly distinguished from its yellow shade in a more acid medium.

The sample, with an arbitrary flow rate, is entered at E and flows through the branch 44, the capillary 42, up to the outlet S. The sample flow rate as issued from a chemical process downstream from the sampling area is 0.5 μL.min⁻¹. When it is desired to obtain a dosage of acidity, the push-syringe 43 is actuated at a flow rate of the order of 10 to 1,000 μL.min⁻¹, and delivers a variable but repeatable amount of dye, typically of the order of 0.5 to 10 μl. A concentration gradient is thereby established in the capillary 42, establishing a more basic blue shaded area and a more acid yellow shaded area. The diode array 41 records the levels of filtered light thereby giving information on the capillary portion 42, opposite each diode, for a given time after stopping the motion of the push-syringe 43. The sample flux may either be stopped or not during the analysis. In this example, the flux is maintained. Measurements issued from the array 41 along the capillary 42 are tracked over time. After calibrating the response for each element, it is possible to give a value of the sample's acidity.

The reagents located in the capillary 42 are then discharged by the flow of the sample. A sufficient sample volume, typically 5 μl or several times the volume of the reaction loop 42, is required in order to avoid a too significant crossover between two analytical measurement sequences.

### SECOND EXAMPLE

### Dosage of Free Acidity in a Loaded Medium

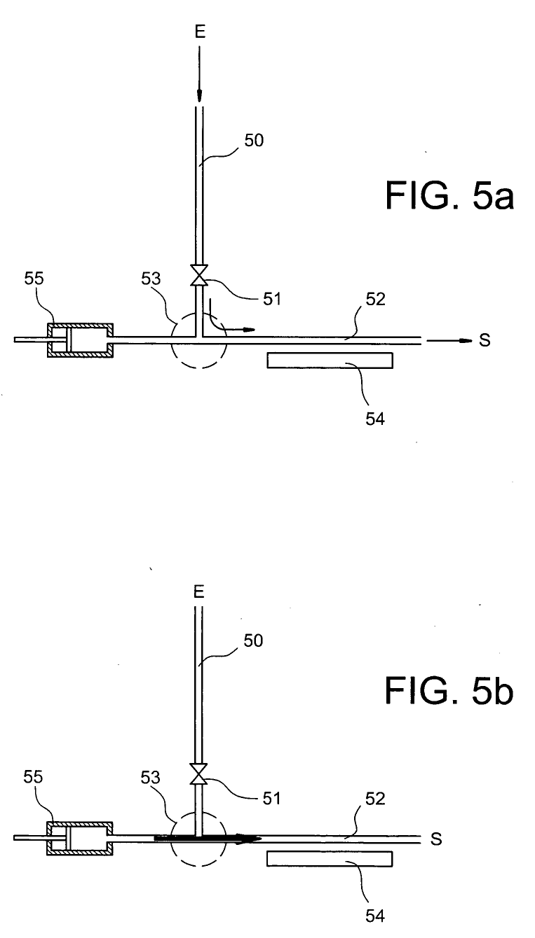

In this example, as illustrated in FIGS. 5a and 5b, the analytical method used is the one for dosing loaded solutions by the oxalate method.

The flow of the sample solution is achieved by gravity and capillarity. A sample of a few tens of microlitres is introduced at the mouth E of a capillary 50 upstream from a valve 51, by a funnel or any other device providing proper filling by capillary and/or gravity over a small volume. The volume of the capillary 50 upstream from the valve 51 is about ten times larger than that of the reaction loop 52. The valve 51, preferably a microvalve, the dead volume of which is much less than the volume of the sample, located upstream from a T-shaped branch 53, allows the sample's flow or the air flow to be stopped when the sample is depleted upstream from the valve 51. The configuration of the other elements is identical with the first example described above, except that the filters used are adapted to the relevant dye. The reagents are soda, a dye changing color around pH 5.5 and a complexing agent, the oxalate. This method differs from the preceding example by the presence of several reagents and by the fact that the sample must be diluted in the reagent by a factor between 20 and 500, in order to provide sufficient complexation of the load.

The flow rate of the sample through the branch 53, the capillary 52 and the detector 54 is arbitrary. It may be discontinuous. It is set by the configuration of the whole.

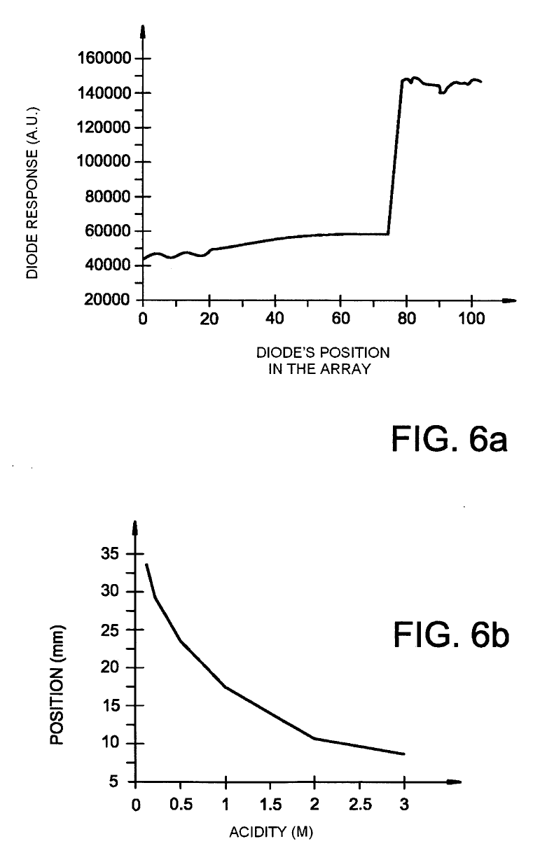

When it is desired to perform the dosage of free acidity, valve 51 is closed in order to prevent any flowing back of the reagents. A first pulse from the push-syringe 55, actuated at a flow rate from 10 to 1,000 μL.min⁻¹, delivers a repeatable and previously fixed amount, typically of the order of 0.5 to 10 μl. A certain waiting time is necessary, typically of the order of 10 seconds, in order to very partially homogenize the mixture depending on the section of the capillary 52. A second identical pulse of reagents again dilutes the sample. Other combinations of pulse/waiting time may then occur in order to take into account the acidities to be analyzed. As in the first example, a concentration gradient is established along the capillary 52. By tracking it over time, it is possible to calculate the value of the acidity. FIG. 6a illustrates a typical plot of values measured on the diode array depending on the position of the diode in the array 54 and therefore on the capillary 52. FIG. 6b illustrates a typical response curve of the position of the color change point in the capillary 52 versus the acidity of the analyte, by taking into account the dispersion of the results over ten measurements made within a time interval of one week between the first and last measurement.

The reagents located in the capillary 52 are then discharged by the flow of the remaining sample, by the flow of gas bubbles separating the samples and by the flow of a portion of the next sample. The total volume of reagents and consumed sample is of the order of 3 to 15 μl.

### THIRD EXAMPLE

### Dosage of a Species Reacting with a Specific Dye, for Example Hydrazine in a Nitric Solution

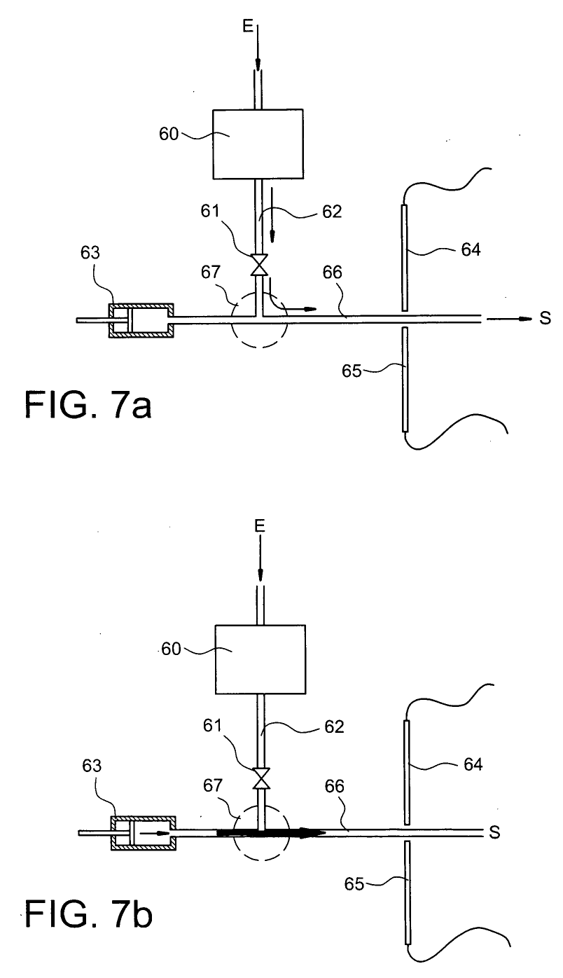

This example, as illustrated in FIGS. 7a and 7b, describes the dosage of hydrazine in a nitric medium by DMAB (dimethylaminobenzaldehyde) so as to measure hydrazine concentrations in acid solution over three decades, of the order of 0.001 to 1 M. This example may also be used for dosages of a species in solution by a reagent when dilutions of the order of 10 to 10,000 are required.

The hydrazine sample is introduced through a peristaltic pump 60 with a flow rate of 100 μL.min⁻¹. A valve 61 is required for isolating the portion upstream from the latter 61 from the T-shaped branch 67, connected to the push-syringe 63 on the one hand, and to the capillary 66 on the other hand. Indeed, in the absence of such a valve 60, upon introducing reagents, the flexible pipes 62 may expand under the sudden pressure surges delivered by the push-syringe 63. This effect would be expressed by a not so good reproducibility of the measurements for time intervals longer than one day. The detector is a point sensor, consisting of two optical fibers 64 and 65 facing each other through the capillary 66, connected to a spectrophotometer and to a light source. The reagent is a DMAB solution of about 0.1 M in 0.5 M nitric acid.

When it is desired to perform an analysis, the peristaltic pump 60 is stopped and the valve 61 is closed. As in the second example, by means of the push-syringe 63, a series of pulses of the reagent is produced at flow rates of the order of 10 to 1,000 μL.min⁻¹ followed by a waiting time. With a spectrophotometer measurement, the absorbance may be checked in order to decide whether a new pulse of reagents is necessary. As soon as the required number of pulses is obtained, absorbance at a point of the capillary 66 is measured versus the elapsed time since the stopping of the last pulse. By preliminary calibration, it is possible to give a value of the hydrazine concentration.

### REFERENCES

[1] U.S. Pat. No. 4,315,754
[2] U.S. Pat. No. 5,849,592
[3] US 2003/032195

1. A method for analyzing a liquid sample by injecting the latter in a reaction loop coupled with illumination means and detection means, characterized in that it comprises the following steps:
filling a reaction loop with a minimum volume of the sample to be analyzed, this reaction loop forming a transparent pipe with which detection means are coupled,
injecting a fixed volume of at least one reagent into the reaction loop,
detecting levels of filtered light by these detection means,
discharging the reagents located in the reaction loop.

2. The method according to claim 1, wherein a concentration gradient is detected in the reaction loop.

3. The method according to claim 1, wherein the reaction loop is a transparent capillary or a microfluidic channel.

4. The method according to claim 1, wherein the discharge of the reagents located in the reaction loop is performed by means of the remaining sample.

5. The method according to claim 1, wherein the discharge of the reagents located in the reaction loop is performed by means of the next sample.

6. The method according to claim 1, wherein the sample flux is not interrupted, which allows continuous analysis.

7. The method according to claim 1, wherein fixed volumes of reagents are successively injected during predefined time intervals.

8. The method according to claim 7, wherein a series of pulses of reagents is produced at flow rates of the order to 10 to 1,000 μl μL.min⁻¹ followed by a waiting time.

9. The method according to claim 1, wherein linear detection is performed along the reaction loop so that it is possible to obtain a space and time plot of the reactions in the set, reaction loop+detection means.

10. The method according to claim 1, wherein a point detection is achieved in a location of the reaction loop so that it is possible to obtain a time plot of the reactions in a location of the set: reaction loop+detection means.

11. The method according to claim 10, wherein a point sensor is used, capable of moving along the reaction loop.

12. A system for analyzing a liquid sample comprising a reaction loop between this sample introduced through an inlet and at least one reagent, and detection means, characterized in that the reaction loop consists of a transparent pipe, and in that said system comprises a push-syringe, the outlet of which is connected to the reaction loop allowing doses of said at least one reagent to be delivered into this loop, and illumination means with which this reaction loop may be illuminated so that the detection means record levels of light transmitted through said loop after filtering.

13. The system according to claim 12, wherein the transparent pipe is a transparent capillary or a microfluidic channel.

14. The system according to claim 12, wherein the detection means comprise a diode array.

15. The system according to claim 12, wherein the detection means comprise two optical fibers positioned on either side of the reaction loop.

16. The system according to claim 12, comprising a peristaltic pump allowing introduction of the sample.

17. The system according to claim 12, comprising a microvalve positioned upstream from the point of introduction of the sample into the reaction loop.

18. The system according to claim 12, wherein a T-shaped branch is respectively connected to the sample inlet, to the push-syringe and to the reaction loop.
